(19) United States
(12) Patent Application Publication
(10) Pub. No.: US 2014/0364764 A1
(43) Pub. Date: Dec. 11, 2014

(54) DEVICE FOR COLLECTING BODY FLUIDS

(71) Applicants: Hyung Il Jung, Seoul (KR); Chengguo Li, Gyeonggi-do (KR); Chang Yoel Lee, Gyeonggi-do (KR); Kwang Lee, Seoul (KR)

(72) Inventors: Hyung Il Jung, Seoul (KR); Chengguo Li, Gyeonggi-do (KR); Chang Yoel Lee, Gyeonggi-do (KR); Kwang Lee, Seoul (KR)

(73) Assignee: INDUSTRY-ACADEMIC COOPERATION FOUNDATION, YONSEI UNIVERSITY, Seoul (KR)

(21) Appl. No.: 14/362,895

(22) PCT Filed: Jul. 19, 2012
(86) PCT No.: PCT/KR2012/005776

§ 371 Date: Aug. 28, 2014

(30) Foreign Application Priority Data
Dec. 13, 2011 (KR) 10-2011-0133655

(51) Int. Cl. A61B 5/151; A61B 5/15

(52) U.S. Cl. CPC A61B 5/15113; A61B 5/150099; A61B 5/150221; A61B 5/150396
USPC 600/579

(57) ABSTRACT

The present invention relates to a device for collecting body fluids, the device comprising: (a) an internal pressure adjustment means for adjusting the internal pressure of the device, and the internal pressure adjustment means has an internal space and is made of an elastic deformable material; (b) a fluid accommodation means, which is openly connected to the internal pressure adjustment means, for accommodating body fluids collected from the human body; and (c) a perforation means which is connected to the fluid accommodation means, is located at the lower part of the device, and comprises a hollow microstructure for forming an opening on the body.

### TECHNICAL FIELD

The present invention relates to a device for extraction of body fluids.

### BACKGROUND ART

The blood is one vital fluid in the human body and plays important roles in the general physiology of human beings. Blood analysis is used to obtain the information about blood physical and chemical properties to monitor human health state. For example, it is extremely important for diabetics to extract blood sample and measure blood glucose level in daily life for prevention and treatment of diabetes. Nowadays, the development of safe, automated and compact real-time systems for blood analysis become one of the most important research themes in the field of medical engineering.

Through blood extraction to get the blood sample is the basic step of detection for diagnosis and remedy of various disease. Generally blood analysis is commonly performed on a blood sample from a vein in the arm using hypodermic needles or via finger-prick. The deficiency in pain due to bigger diameter of hypodermic needles and immunization reaction has been the main problem in the development of real-time blood analysis systems such as ubiquitous healthcare, point of care and health monitoring device. Using microneedles to extract blood can overcome these limitations due to small size and biocompatible material used for fabrication. During the past years, people have put into considerable effort to develop minimally invasive and biocompatible hollow microneedles for blood extraction to replace the traditional hypodermic needles.

A number of miniature blood extraction devices has been successfully fabricated and are widely used in the health monitoring systems by the development of Bio-MEMS (Biomedical-Micro Electro Mechanical Systems). The microneedles are so far fabricated from silicon[1], plastics[2] and metals[3]. The term “electronic mosquito”[4] suggests that use the silicon microneedle in blood analysis system. While the length of silicon microneedle is not viable for blood extraction, it is required to reach into the deeper layers of the dermis at around 1500 um to be in touch with the blood vessels and it also has the risks of breakage inside the skin. Though some plastics and metals microneedles which are fabricated using the deep X-ray lithography of LIGA (Lithographie, Galvanoformung, and Abformung) technique [8] and sputtering deposition method [5] are also widely used in the blood extraction system, in comparison might be biocompatible and have a good stiffness, the way of fabrication is too complicated and costly to mass-produce. It is necessary to design and fabricate the minimally invasive hollow microneedle to be stiff enough to insert into skin and have suitable length to penetrate the capillary vessel extraction of blood.

Another important part of blood extraction device is micropumps which are the power provider for blood extraction. Many kinds of actuators are used to fabricate micropumps e.g. piezoelectric[5], electrostatic[6], SMA (Shape Memory Alloy)[7] and vacuum drive system [8]. However, the blood extraction flow rate of the piezoelectric and electrolysis drive micropumps is too slow and fabrication are complicated. SMA actuators not only needs a long cooling time but also their response time is too slower than other micropump actuators and the most important is the bigger size not suitable for disposal. All the reported micropumps need the external power source and they were also costly with complicated fabrication method. On the other hand, it is difficult for the vacuum drive system to transport the blood sample to other analysis devices. Many factors make these blood extraction devices not suitable for disposal and single uses by patients.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### DETAILED DESCRIPTION OF THE INVENTION

### Technical Problem

The present inventors endeavored to develop a device for extraction of body fluids, capable of extracting body fluids (preferably, blood) from a subject (preferably, humans) painlessly, with minimum injury, and at improved efficiency. As a result, the present inventors developed a device for extraction of body fluids, capable of being convenient to use, portable, and efficient in extracting body fluids, and thus the present inventors completed the present invention.

Accordingly, an aspect of the present invention is to provide a device for extraction of body fluids.

Another aspect of the present invention is to provide an integrated analysis system of body fluids.

Other purposes and advantages of the present disclosure will become clarified by the following detailed description of invention, claims, and drawings.

### Technical Solution

In accordance with an aspect of the present invention, there is provided a device for extraction of body fluids, the device including: (a) an internal pressure regulator made of an elastically deformable material and having an internal space therein, the internal pressure regulator regulating the internal pressure of the device; (b) a fluid reservoir openly connected to the internal pressure regulator and receiving body fluids extracted from the body; and (c) a perforator connected to the fluid reservoir and disposed at a lower portion of the device, the perforator including a hollow microstructure that forms a hole in the body.

The present inventors endeavored to develop a device for extraction of body fluids, capable of extracting body fluids (preferably, blood) from a subject (preferably, humans) painlessly, with minimum injury, and at improved efficiency. As a result, the present inventors developed a device for extraction of body fluids, capable of being convenient to use, portable, and efficient in extracting body fluids, and thus the present inventors completed the present invention.

The device for extraction of body fluids of the present invention will be described with reference to the accompanying drawings.

The device of the present invention includes an internal pressure regulator 1 for regulating the internal pressure of the device. The internal pressure regulator 1 is made of an elastically deformable material and has an internal space therein. The internal pressure regulator 1 serves to produce a negative pressure for extracting a fluid sample.

According to a preferable embodiment of the present invention, the device of the present invention further includes an outlet valve connected to the fluid reservoir and an inlet valve provided between the fluid reservoir and the hollow microstructure. These check valves precisely control the inflow and outflow of a fluid.

The internal pressure 1 is openly connected to a fluid reservoir 2. The change in pressure, which occurs in the internal pressure regulator 1, induces a fluid to be sucked into the fluid reservoir 2 from the hollow microstructure. In one embodiment of the present invention in which the outlet valve 21 and the inlet valve are absent (FIG. 1a), the change in pressure, which occurs in the internal pressure regulator 1, induces a fluid to be directly sucked into the fluid reservoir 2 from the hollow microstructure. In an embodiment of the present invention in which the outlet valve 21 and the inlet valve 22 are present (FIG. 1b), the change in pressure, which occurs in an internal pressure regulator 1, is transferred to the outlet valve 21 and the inlet valve 22, thereby controlling the opening and closing of the outlet valve 21 and the inlet valve 22 and inducing a fluid to be sucked into the fluid reservoir 2 from the hollow microstructure.

The internal pressure regulator 1 is made of an elastically deformable material, such that the internal pressure regulator 1 is deformed from its shape by an external pressure, thereby controlling the internal pressure of the device. The external pressure applied to the device of the present invention is preferably a pressure that is applied by a repulsive force by finger press. The internal pressure regulator 1 produces a negative pressure by an elastic deformation force.

The elastically deformable material used in fabricating the internal pressure regulator 1 includes any elastically deformable material known in the art. According to a preferable embodiment, the elastically deformable material used in the present invention is a silicon polymer or copolymer, an epoxy polymer or copolymer, or an acrylic polymer or copolymer. The elastically deformable material used in the present invention is a polymer, which may have a linear or branched chain backbone and may be cross-linked or non-cross-linked.

The epoxy polymer usable in the present invention is characterized by the presence of 3-membered cyclic ether group known as an epoxy group. For example, bisphenol A diglycidyl ether or the like may be used in the present invention.

A silicone elastomer usable in the present invention is a polymer formed from a precursor such as chlorosilane (e.g., methyl chlorosilane, ethyl chlorosilane, and phenyl chlorosilane). A particularly preferable silicon polymer is polydimethyl siloxane (PDMS). An exemplary polydimethyl siloxane polymer is purchasable from Dow Chemical Inc. under the product name of Sylgard. Specifically, Sylgard 182, Sylgard 184, and Sylgard 186 are suitable.

As described above, in one embodiment of the present invention in which the outlet valve 21 and the inlet valve are absent (FIG. 1a), the change in pressure in the internal pressure regulator 1 induces a fluid to be directly sucked into the fluid reservoir 2 from the hollow microstructure.

According to a preferable embodiment of the present invention, the internal pressure regulator 1 is deformed from its shape by a downward external pressure, resulting in reducing the volume of the internal space, thereby applying a perforation force to allow the hollow microstructure 31 in contact with a body surface barrier to perforate the body surface barrier.

According to a preferable embodiment of the present invention, the internal pressure regulator 1 is restored to its original shape by the relaxing of the external pressure, resulting in producing a negative pressure inside the device, thereby allowing a fluid to flow into the fluid reservoir 2 from the hollow microstructure 31 in contact with the body surface barrier.

According to a preferable embodiment, the internal pressure regulator 1 after the relaxing of the external pressure is deformed from its shape by an application of a downward external pressure, resulting in reducing the volume of the internal space and thus increasing the internal pressure of the internal space, thereby allowing the fluid reserved in the fluid reservoir 2 to flow out through the hollow microstructure 31 (FIG. 5a).

As described above, in one embodiment of the present invention in which the outlet valve 21 and the inlet valve are present (FIG. 1b), the change in pressure in the internal pressure regulator 1 is interworked with the opening and closing of the outlet valve 21 and the inlet valve 22.

According to a preferable embodiment of the present invention, the internal pressure regulator 1 is deformed from its shape by a downward external pressure, resulting in reducing the volume of the internal pressure and thus increasing the internal pressure of the device, thereby inducing the opening of the outlet valve 21 and the closing of the inlet valve 22 and applying a perforation force to allow the hollow microstructure in contact with a body surface barrier (preferably, human skin) to perforate the body surface barrier (FIG. 5b).

According to a preferable embodiment of the present invention, the internal pressure regulator 1 is restored to its original shape by the relaxing of the external pressure, resulting in producing a negative pressure inside the device and thus closing the outlet valve 21 and opening of the inlet valve 22, thereby allowing a fluid to flow into the fluid reservoir 2 from the hollow microstructure 31 in contact with the body surface barrier (preferably, human skin) through the opened inlet valve 22 (FIG. 5b).

According to a preferable embodiment of the present invention, the internal pressure regulator 1 after the relaxing of the external pressure is deformed from its shape by an application of a downward external pressure, resulting in reducing the volume of the internal space and thus increasing the internal pressure of the internal space, thereby inducing the opening of the outlet valve 21 and the closing of the inlet valve 22 and allowing a fluid (preferably, blood) reserved in the fluid reservoir 2 to flow out through the outlet valve 21 (FIG. 5b).

According to a preferable embodiment of the present invention, the outlet valve 21, the inlet valve 22, or both of the outlet valve 21 and the inlet valve 22, which are installed at the device, have a pneumatic flap valve type. Preferably, both of the outlet valve 21 and the inlet valve 22 are pneumatic flap valves. That is, the outlet valve 21 and the inlet valve 22 are pneumatic flap valves of which the opening and closing are controlled by an air pressure (regulated by the internal pressure regulator).

The internal pressure regulator 1 may have various shapes, and may be fabricated in various manners. For example, the internal pressure regulator 1 may consist of an upper part having a cuboidal shape with an internal space therein and a lower part 12 having a cylinder structure 12a that protrudes by a predetermined height. The cylinder structure 12a has the same diameter as a hollow cylinder portion of the fluid reservoir 2 such that the internal pressure regulator 1 is easily coupled with the fluid reservoir 2. A hollow space is formed in the middle of the cylinder structure 12a such that a channel is formed in the cylinder structure 12a. Thus, the internal regulator 1 and the fluid reservoir are openly connected with each other.

In the device of the present invention, the fluid reservoir 2 is a reservoir of a fluid, especially, blood that is extracted. The fluid reservoir 2 is also preferably made of an elastically deformable material. A hollow space with a predetermined diameter is formed in a lateral surface of the fluid reservoir 2 such that the fluid reservoir 2 communicates with the outlet valve.

In the device of the present invention, the outlet valve 21 controls the outflow of the fluid extracted in the device to the outside. The outlet valve 21 may be fabricated in various manners, and preferably has an in-contact flap-stopper structure (FIG. 4). According to a preferable embodiment of the present invention, the in-contact flap-stopper structure includes: (i) an outlet valve flap plate 211 having a flap 211a that is openable and closable; and (ii) a stopper plate 212 closely adhering to the outlet valve flap plate 211, wherein a pore of the stopper plate 212 communicates with the flap 211a of the outlet valve flap plate 211 when the flap 211a is opened and the fluid reservoir 2. The pore of the stopper plate 212 of the outlet valve 21 communicates with the hollow space formed in the lateral surface of the fluid reservoir 2. In the above structure, the outlet valve 21 can strongly adhere to the fluid reservoir 2 and increase the efficiency of blood extraction. In the in-contact flap-stopper structure of the outlet valve 21, the lateral surface with the hollow space in the fluid reservoir 2 may serve as the stopper plate 212. In this case, the outlet valve flap plate 211 is attached to the lateral surface of the fluid reservoir 2 without the stopper plate 212.

In the device of the present invention, the inlet valve 22 controls the inflow of the fluid from the hollow microstructure to the fluid reservoir 2. The inlet valve 22 may be fabricated in various manners, and preferably has a not-contact flap-stopper structure (FIG. 3). The not-contact flap-stopper structure includes: (i) an inlet valve flap plate 221 having a flap that is openable and closable; (ii) a stopper plate 223 having a pore communicating with the hollow microstructure; and (iii) an intermediate plate 222 disposed between the inlet valve flap plate and the stopper plate, wherein a pore of the intermediate plate communicates with the flap of the inlet valve flap plate 221 when the flap is opened and the pore of the stopper plate. The above-described not-contact flap-stopper structure of the inlet valve is favorable in extracting blood at high efficiency under even a low negative pressure. The inlet valve 22 of the device of the present invention is easily opened or closed, and exhibits a low leakage rate of blood when the blood is transported from the fluid reservoir 2 to another part, for example, the outside.

The device of the present invention includes a perforator 3 connected to the fluid reservoir 2 and positioned at a lower portion of the device. The perforator 3 includes a hollow microstructure that forms a hole in the body. Preferably, the perforator 3 may include a hollow microstructure 31 and a support 32 supporting the hollow microstructure 31 (see: FIG. 2).

The hollow microstructure used in the present invention may include any hollow microstructure known in the art. Preferably, the hollow microstructure used in the present invention is a hollow microstructure for minimally invasive blood extraction, which was developed by the present inventors and disclosed in Korean Patent Application No. 2011-0078510. Thus, details of the hollow microstructure used in the present invention are given in the disclosure of Korean Patent Application NO. 10-2011-0078510.

According to a preferable embodiment of the present invention, the hollow microstructure used in the present invention is a hollow microstructure for minimally invasive blood extraction that has a length of 1-5000 μm, an inner diameter of 10-100 μm, a bevel angle of 5-60°, a tip angle of 1-45°, and a tip transverse length of 2-30 μm. These dimensions of the hollow microstructure are most appropriate in extracting blood from a subject (preferably, humans) painlessly, with minimum injury, and at improved efficiency, and the present inventors constructed these dimensions.

As used herein, the term “inner diameter” of the hollow microstructure, unless otherwise particularly specified, refers to an inner diameter of an upper end portion (the minimum-diameter end portion of the microstructure). The inner diameter of the hollow microstructure is preferably 10-100 μm, more preferably 20-80 μm, still more preferably 30-70 μm, and still more preferably 50-70 μm. The length of the hollow microstructure is preferably 200-5000 μm, more preferably 1000-4000 μm, still more preferably 1200-3000 μm, still more preferably 1500-2500 μm, and most preferably 200-2200 μm. The hollow microstructure has a tip transverse length of preferably 2-30 μm. In addition, the hollow microstructure used in the present invention has a tip angle of preferably 1-45°. The term “tip”, used when the hollow microstructure is cited herein, refers to a tip region of the upper end portion of the microstructure, to which the bevel angle is given. The term “tip end portion” refers to, when the bevel angle is given to the tip region of the upper end portion of the microstructure such that a hollow space is seen from the outside, a region from the top end of the hollow space to the furthest end of the microstructure (see: FIG. 11). The term “tip transverse length” refers to a length of the line across the tip end portion at the middle region of the tip end portion (see: FIG. 11). The term “tip angle” refers to an angle between both edges at the tip end portion (see, FIG. 11). The present inventors recognized the problem in which the microneedle having a tip region with a bevel angle cannot satisfy the minimum invasion. The existing technologies did not achieve the minimum invasion since the end of the tip is relatively large due to the application of simple bevel. However, according to the present invention, the tip end portion of the microneedle was polished such that the tip transverse length was 2-30 μm (preferably, 2-10 μm, 5-10 μm, and 2-8 μm) and the tip angle was 1-45° (preferably, 30-45°).

The hollow microstructure used in the present invention is preferably a microneedle, a microblade, a microknife, a microfiber, a microspike, a microprobe, a microbarb, a microarray, or a microelectrode; more preferably a microneedle, a microblade, a microknife, a microfiber, a microspike, a microprobe, or a microbarb; and most preferably a hollow microneedle.

In the perforator 3, the support 32 supporting the hollow microstructure 31 may serve as the stopper plate 223 of the inlet valve 22. In this case, the inlet valve 22 consists of the inlet valve flap plate 221 and the intermediate plate 222, and the diameter of the support 32 of the perforator 3 is preferably the same as the diameter of the hollow space of the middle plate 222.

The body fluid extracted by the device of the present invention includes various fluids such as blood, interstitial fluid, eyeball fluid, and the like. The body fluid is preferably blood, and more preferably human blood.

In accordance with another aspect of the present invention, there is provided an integrated analysis system of body fluids, the system including the above-described device for extraction of body fluids; and an analysis device of the body fluids.

The analysis device used in the integrated analysis system of the present invention includes, but is not limited to, a microarray (or a microchip), a biosensor, an immune chromatography-based analysis device (e.g., rapid kit), an ELISA kit, a polymerase chain reaction (PCR) analysis device, and a real-time PCR analysis device, which have a probe with a microchannel or an antibody.

### Advantageous Effects

Features and advantages of the present invention are summarized as follows:

(a) The present invention provides a device for fluid extraction, capable of extracting body fluids (preferably, blood) from a subject (preferably, humans) painlessly, with minimum injury, and at improved efficiency.

(b) The device of the present invention can be easily fabricated, conveniently operated, and portable, and can efficiently extract fluids.

(c) The device for fluid extraction of the present invention is incorporated with an analysis device, thereby efficiently analyzing the fluid, especially, blood.

### BRIEF DESCRIPTION OF THE DRAWINGS

FIG. 1a shows a device for fluid extraction in which an outlet valve and an inlet valve are absent according to an embodiment of the present invention. 1: internal pressure regulator; 2: fluid reservoir; 3: perforator; 31: hollow microneedle.

FIG. 1b shows a device for fluid extraction in which an outlet valve and an inlet valve are present according to an embodiment of the present invention. 1: internal pressure regulator; 2: fluid reservoir; 3: perforator; 21: outlet valve; 22: inlet valve; 31: hollow microneedle

FIG. 2 illustrates respective components of a device for fluid extraction of the present invention. 11: cuboidal shaped upper part of internal pressure regulator; 12: lower part of internal pressure regulator; 12a: cylinder structure of lower part of internal pressure regulator; 2: fluid reservoir; 21: outlet valve; 221a: flap of outlet valve flap plate; 221: inlet valve flap plate; 221a: flap of inlet valve flap plate; 222: intermediate plate; 3: perforator; 31: hollow microstructure; 32: support

FIG. 3 shows a not-contact flap-stopper structure of an inlet valve in a device of the present invention. 22: inlet; 221: inlet valve flap plate; 221a: flap of inlet valve flap plate; 222: intermediate plate; 223: stopper plate.

FIG. 4 shows an in contact flap-stopper structure of an outlet valve in a device of the present invention. 21: outlet valve; 211: outlet valve flap plate; 212: stopper plate

FIG. 5a is a schematic view showing an operating principle of the device for fluid extraction in which an outlet valve and an inlet valve are absent according to an embodiment of the present invention.

FIG. 5b is a schematic view showing an operating principle of the device for fluid extraction in which an outlet valve and an inlet valve are present according to an embodiment of the present invention.

FIG. 6 shows results of negative pressures for different volumes (81 μl, 162 μl, 243 μl, 324 μl, and 405 μl) of an internal pressure regulator in a device of the present invention, which were measured using a mamometer. The wording “PDMS bulb” represents an internal pressure regulator.

FIG. 7 shows results for distilled water (DW), blood-mimicking fluid (BMF), and human blood, as a fluid, which were extracted by using a device of the present invention. The wording “PDMS bulb” represents an internal pressure regulator.

FIG. 8 shows images in which mouse blood was extracted by using a device of the present invention.

FIG. 9 is a schematic view showing an integration of a device for fluid extraction of the present invention and a diagnostic kit.

FIG. 10 is a schematic view showing an integration of a device for fluid extraction of the present invention and a microchip.

FIG. 11 shows a hollow microneedle in a device for fluid extraction of the present invention.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Example 1

### Fabrication of Hollow Microneedle for Minimally Invasive Blood Extraction

A solid microneedle was fabricated by using SU-8 2050 photoresist (purchased from Microchem) having a viscosity of 14,000 cSt. The SU-8 2050 negative photoresist was coated onto metal and silicon substrates to 1000 μm and 2000 μm, respectively, and then kept at 120 for 5 minutes to maintain fluidity of SU-8. The photoresist was then placed in contact with a prepared 3×3 pattern frame having a diameter of 200 μm. While the temperature of the substrate was slowly lowered to 70 to 60, the coated SU-8 2050 photoresist has such a viscosity that it can be lifted. Here, the lifting frame was lifted at a speed of 10 μm/s for 5 minutes, thereby fabricating an initial solid structure of 3,000 μm. The formed initial solid structure may be separated from the lifting frame by increasing the speed of a second lifting or performing a cutting process. As a result, the initial coating thickness of 1,000 μm resulted in a solid microstructure having an upper end diameter of 30 μm, a lower end diameter of 200 μm, and a length of 1,500 μm, and the initial coating thickness of 2,000 μm resulted in a solid microstructure having an upper end diameter of 40 μm, a lower end diameter of 300 μm, and a length of 2,000 μm. Chemical deposition was conducted by the Tollen's reaction. Then, the upper end of the solid microneedle was protected by enamel or SU-8 2050. The treatment of the upper end with enamel or SU-8 2050 is for preventing the upper end from being plated in a subsequent step. After metal plating of the entire solid microstructure, laser cutting or microsawing may be performed to allow the solid microstructure to have a hollow type. Then, the surface of the solid microneedle with the protected upper end was electroplated with nickel. The nickel electroplating was performed at 0.206 μm/min for 1 A/dm² for 75 minutes, so that the plating metal thickness was 20 μm. Subsequently, the upper end of each of the metal-plated solid microstructures was cut vertically (at an angle of 0°), at an angle of 75°, 45°, 60°, or 15°. Then, the structure was inserted in the SU-8 remover (purchased from Microchem) at 60 to 100 for 1 hour to remove the solid microstructure of SU-8 2050, thereby fabricating a hollow microneedle. Then, the end of the tip of the hollow microneedle was cut in three directions such that the tip transverse length was 10 man or 8 μm, thereby finally fabricating a hollow microneedle for minimally invasive blood extraction.

With regard to the fabricated hollow metallic microneedles for minimally invasive blood extraction, the initial coating thickness of 1,000 μm resulted in a hollow microneedle having an outer diameter of 70 μm and an inner diameter of 30 μm at the upper end, a diameter at the lower end of 200 μm, and a length of 1,500 μm, and the initial coating thickness of 2,000 μm resulted in a hollow microneedle having an outer diameter of 100 μm and an inner diameter of 60 μm at the upper end, a diameter at the lower end of 200 μm, and a length of 1,500 μm. Strength values of the fabricated hollow microneedles showed 1-2 N, which are stronger than the strength value to penetrate the skin, 0.06N.

The hollow metallic microneedles for minimally invasive blood extraction were fabricated by the same method as above except for slight changes of conditions. The fabricated hollow metallic microneedles had different inner diameters, bevel angles, tip transverse lengths, and tip angles depending on the fabrication conditions thereof.

### Example 2

### Blood Extraction Using Hollow Microneedle for Minimally Invasive Blood Extraction

Influence of Change in Inner Diameter of Hollow Microneedle at the Time of Actual Blood Extraction

The syringe was placed vertically on the syringe pump. A pressurizer was connected to the end of the syringe, and then slowly drawn to produce a negative pressure. Then, the negative pressures at a predetermined volume were measured by a pressure gauge. The average thereof was obtained and then determined as the standard of a negative pressure. Under the conditions of the same negative pressure (P=15.44 kPa), the blood extraction volumes by hollow microneedles with various sized inner diameters were measured (Table 1). As a result of experiment, blood was not able to be extracted due to a blockage phenomenon when the inner diameter was 50 μm or smaller. The blockage phenomenon was significantly reduced when the inner diameter was 70 μm, and the blockage phenomenon did not occur when the inner diameter was 80 μm or larger. In addition, it can be seen that, when the inner diameter was 60 μm or larger, the increase in the inner diameter led to an increase in the blood extraction rate and an increase in the blood extraction volume.

TABLE 1
Actual blood extraction rate and possibility of blockage

| | Inner diameter of microneedle (μm) | | | | |
|---|---|---|---|---|---|
| | 40 | 50 | 60 | 70 | 80 |
| Blood extraction rate (μl/s) | No | No | 1.69 | 2.67 | 2.89 |
| Possibility of blockage (%) | 100 | 90 | 80 | 10 | 0 |

Influence of Change in Bevel Angle on Actual Blood Extraction

In order to achieve the minimal invasion and minimize the microneedle blockage, the bevel angle was given to the end of the hollow microneedle. In order to analyze the flow of blood fluid, including influence of blood cells, the blood sample of an experimenter was treated with EDTA (a chemical anticoagulant). In the above experiment, since the microneedle with a 60 μm-inner diameter showed the possibility of blood extraction, the bevel angle is given to the end of the microneedle with a 60 μm-inner diameter by using a laser, so that the influence of the bevel on the blockage was observed. Various bevel angles (90°, 45°, and 15°) were applied to hollow microneedles having the same negative pressure (0.337 kPa/s) and the same inner diameter (60 μm), so that the blockage phenomenon at the time of blood extraction was measured (treatment of blood with EDTA, 20 experiments for each bevel angle). As an experimental result, the smaller bevel angle further moderated the blockage. Throughout the present experiment, the 15° bevel angle was decided to be applied to the hollow microneedle.

Determination on Conditions of Hollow Microneedle for Optimal Blood Extraction

Based on the experiment results, a length of 2000 μm, an inner diameter of 60 μm, an outer diameter of 120 μm, a bevel angle of 15°, a tip transverse length of 10 μm (or 8 μm), and a tip angle of 30-45° were determined for the optimum hollow microneedle for minimally invasive blood extraction. In order to prevent the blockage of the microneedle by the blood and improve the efficiency of blood extraction, it is preferable to use several microneedles simultaneously at the time of blood extraction.

### Example 3

### Fabrication of Device for Fluid Extraction (Blood Extraction)

Principle of Device for Blood Extraction of Present Invention

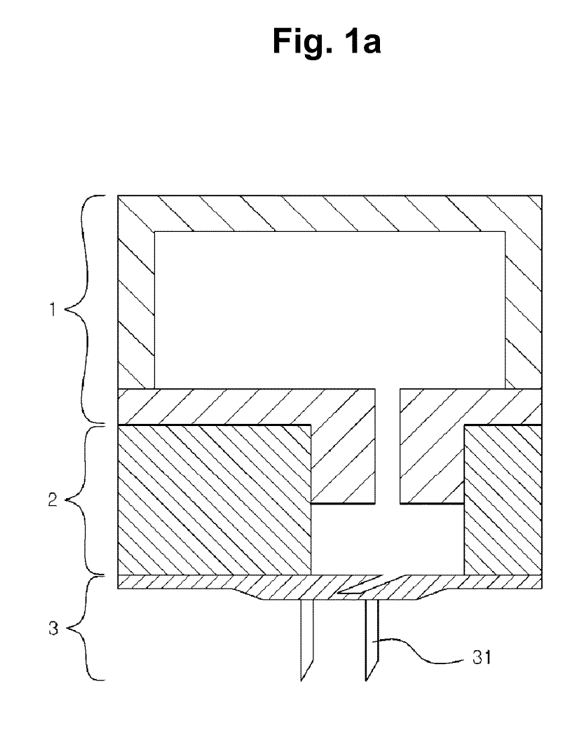

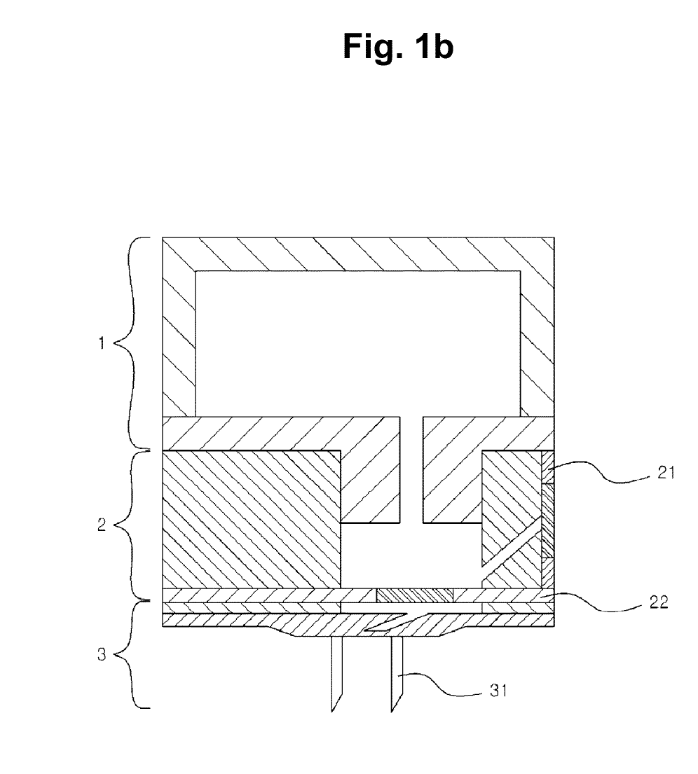

The present inventors developed a painless and portable device for blood extraction (height: 11 mm, width: 11 mm). The device for blood extraction of the present invention is largely composed of three parts (see: FIG. 1b). (a) an internal pressure regulator (1) made of a highly elastically deformable material (e.g., PDMS) and producing a negative pressure for extracting a blood sample; (b) a fluid reservoir 2 (preferably made of PDMS) equipped with two passive check valves (an inlet valve 21 and an outlet valve 22) for controlling the blood sample to be extracted and transported to another part; and (c) a perforator 3 connected to the fluid reservoir 2 and disposed at a lower portion of the device, the perforator 3 including a minimally invasive hollow microneedle 31 for forming a hole in the body.

The device for blood extraction of the present invention was fabricated by using a highly elastically deformable polymer, PDMS. The device of the present invention can be conveniently fabricated at low costs without any electrical/electronic element, battery, or power supplier, and requires only the repulsive force by finger press. When, after the penetration of the hollow microneedle into the skin through the finger force, the PDMS internal pressure regulator 1 is pressed, and then the finger force is relaxed, the blood sample flows into the fluid reservoir 2 by the negative pressure formed by an elastic deformation force of the PDMS internal pressure regulator. The deformation and deformation force of the PDMS internal pressure regulator are preferably axial deformation and deformation force.

The device for fluid extraction of the present invention is specifically named as “device for blood extraction”, “PDMS hand pump”, or “PDMS blood extraction device” in the examples.

Fabrication of Device for Blood Extraction of Present Invention

(a) Fabrication of Internal Pressure Regulator 1 and Fluid Reservoir 2

For the fabrication of PDMS hand pump of the present invention, the internal pressure regulator 1 and the fluid reservoir 2 were fabricated by the traditional micro-fabrication technique. The two kinds of check valves were fabricated using the sandwich molding process [9,10].

For fabrication of the parts of PDMS pump of the present invention, the curing agent and PDMS prepolymer (Dow Corning, MI, USA) were mixed in 1:10 weight ratio. The prepolymer mixture was poured onto the masters and cured at 80° C. for 3 hour. The resulting PDMS layers were peeled off from the masters and later were assembled with each other.

More specifically, two masters of the PDMS internal pressure regulator 1 were placed in the petri dish, and then a PDMS prepolymer mixture was poured onto the masters. As a result, a hollow PDMS cuboid 11 having a thickness of 1 mm, a length of 11 mm, a width of 11 mm, a height of 4 mm, and an inside volume of 243 μl was prepared. Another part of the PDMS internal pressure regulator 1 is a bottom PDMS layer 12 with a cylinder structure 12a that protrudes by 2 mm. The cylinder structure 12a had a diameter of 4 mm, which is the same as that of a hollow cylinder part of the fluid reservoir 2. This cylinder structure allows the internal pressure regulator 1 to be easily coupled with the fluid reservoir 1. The two parts of the PDMS internal pressure regulator 1 were coupled with each other. Finally, a hollow space with a 2-mm diameter is formed in the middle portion of the cylinder structure 12a such that a channel is formed in the cylinder protrusion 12a, thereby completing the PDMS internal pressure regulator 1.

The PDMS fluid reservoir 2 was fabricated by pouring the PDMS prepolymer mixture into the mold of PDMS fluid reservoir 2 in accordance with the same method as the internal pressure regulator 1. The side of PDMS fluid reservoir 2 was punctured to form a puncture in 1-mm diameter using a blunt-end punch to be openly connected to the outlet valve

(b) Fabrication of Outlet Valve and Inlet Valve

The PDMS hand pump of the present invention has two kinds of check valves (outlet 21 and inlet valves 22). The check valves were fabricated using sandwich molding process [9,10].

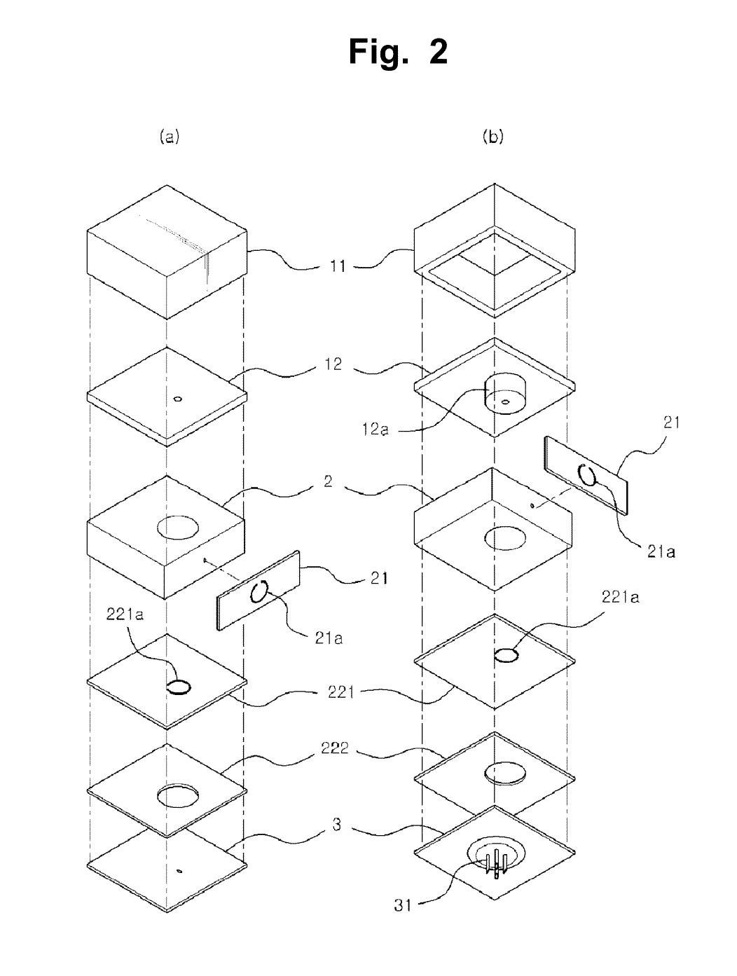

More specifically, the inlet valve 22 was fabricated by the following scheme. When there is a negative pressure in the fluid reservoir 2, it is preferable to use a low pressure in a pore of the inlet valve 22 to increase the efficiency of blood extraction. Generally, the longer the distance between a flap and a stopper, the higher the extraction rate while the shorter the distance therebetween, the lower the extraction rate. As shown in FIG. 3, the inlet valve 22 was fabricated in a not-contact flap-stopper structure, which is favorable in blood extraction at high efficiency even in the low negative pressure. The not-contact flap-stopper structure includes: (i) an inlet valve flap plate 221 having a flap 221a that is openable and closable; (ii) a stopper plate 223 having a pore communicating with the hollow microstructure; and (iii) an intermediate plate 222 disposed between the inlet valve flap plate 221 and the stopper plate 223, a pore of the intermediate plate 222 communicating with the flap 221a of the inlet valve flap plate 221 when the flap 221a is opened and the pore of the stopper plate 221. The distance between the inlet valve flap plate 221 and the stopper plate 223 was 100 μm, and the thickness of the inlet valve flap plate 221 was 100 μm. The stopper plate 223 was fabricated by the PDMS layer connected with the hollow microneedle. This inlet valve 22 is easily opened or closed, and exhibits a low leakage rate of the blood when the blood is transported from the fluid reservoir 2 to another part, for example, the outside.

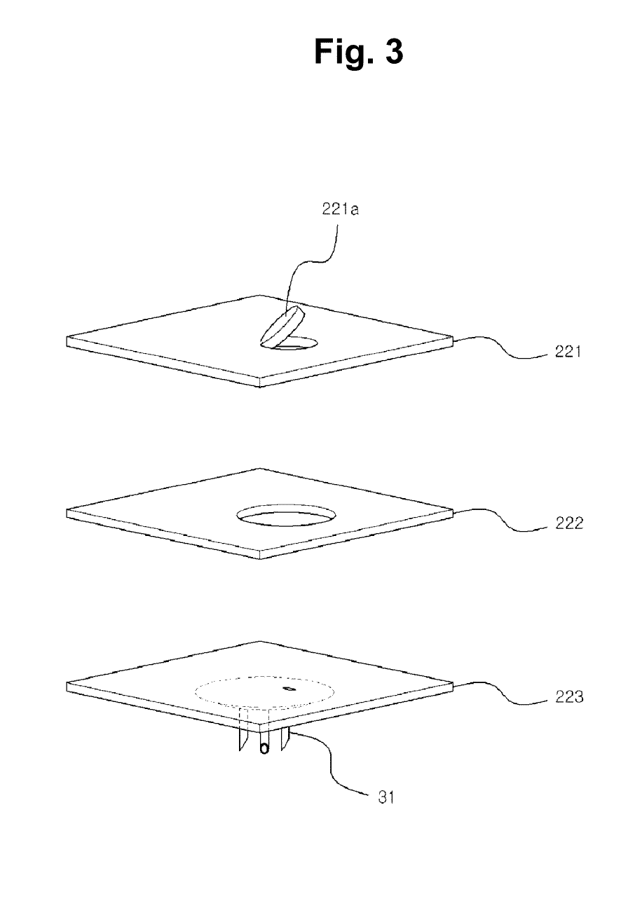

The outlet valve 21 was fabricated by the following scheme (FIG. 4). When the negative pressure is produced in the device of the present invention, the outlet valve 21 should be very strongly coupled with the fluid reservoir 2 to maintain the negative pressure of the fluid reservoir 2. The outlet valve 21 has an in-contact flap-stopper structure¹², so that an outlet valve flap plate 211 strongly adhere to a stopper plate 212. The outlet valve flap plate 2111 and the stopper plate 212 in the outlet valve 21 were fabricated in the same manner as the inlet valve flap plate 221 and the stopper plate 223 in the inlet valve 22. The pore of the stopper plate 212 of the outlet valve 21 communicates with the hollow space formed in the lateral surface of the fluid reservoir 2. In this structure, the outlet valve 21 can strongly adhere to the fluid reservoir 2 and increase the efficiency of blood extraction.

All the parts of the device for blood extraction, which were fabricated as above, were assembled. Then, the PDMS surfaces were activated by using oxygen plasma, and then strongly bonded, thereby finally completing the device for blood extraction of the present invention.

### Example 4

### Operation of Device for Fluid Extraction (Blood Extraction)

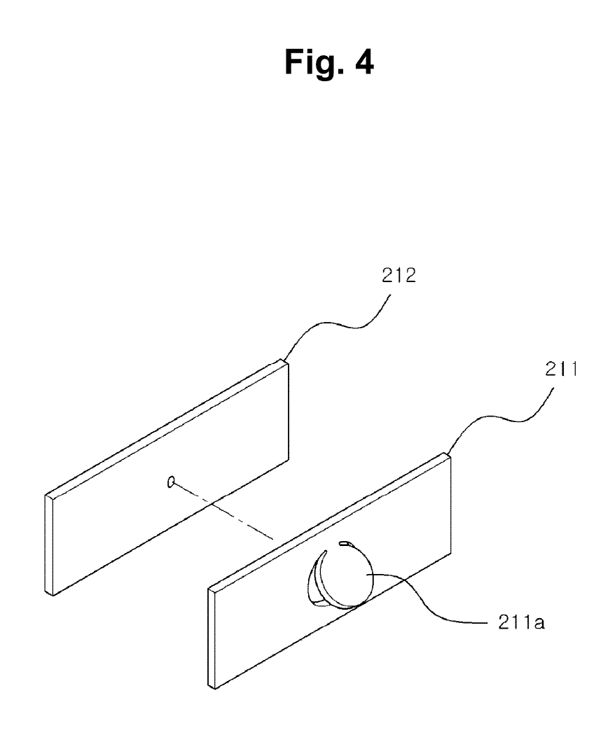

FIG. 5b shows an operating principle of a device for blood extraction of the present invention in which an outlet valve and an inlet valve are present.

(A) First Step

When the internal pressure regulator 1 of the elastically deformable PDMS bulb is pressed, the inlet valve 22 is closed and the outlet valve 21 is opened. The air in the internal pressure regulator 1 is driven out via the outlet valve 21 and the microneedles 31 are inserted into skin by the compression force.

(B) Second Step

Upon relaxing the pressure force applied to the internal pressure regulator 1 of the PDMS bulb, the internal pressure regulator 1 is restored to its original shape by its high elastic deformation force. The internal pressure regulator 1 of the PDMS bulb produces the negative pressure and permits to extract blood into the fluid reservoir 2 with closing the outlet valve 21 by the pressure difference between inside of the fluid reservoir 2 and outside of the chamber.

(C) Third Step

After pulling out the hollow microneedles 31 from skin and pressing again the internal pressure regulator 1 of the PDMS bulb, the inlet valve 22 is closed and the outlet valve 21 is opened and blood reserved in the fluid reservoir 2 is transported to outside of the device.

### Example 5

### Blood Extraction Using Device for Fluid Extraction (Blood Extraction)

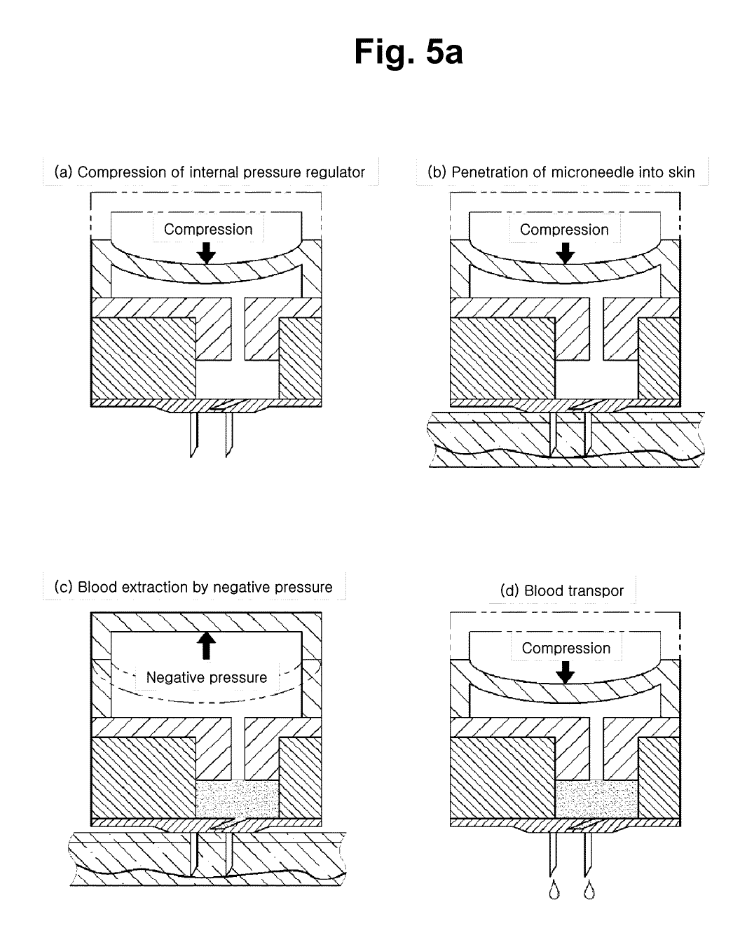

In devices for blood extraction of the present invention, the negative pressures formed by using internal pressure regulators with different volumes (81 μl, 162 μl, 243 μl, 324 μl, and 405 μl) were measured using a mamometer. As can be confirmed from FIG. 6, the negative pressure was increased in proportion to the volume of the internal pressure regulator.

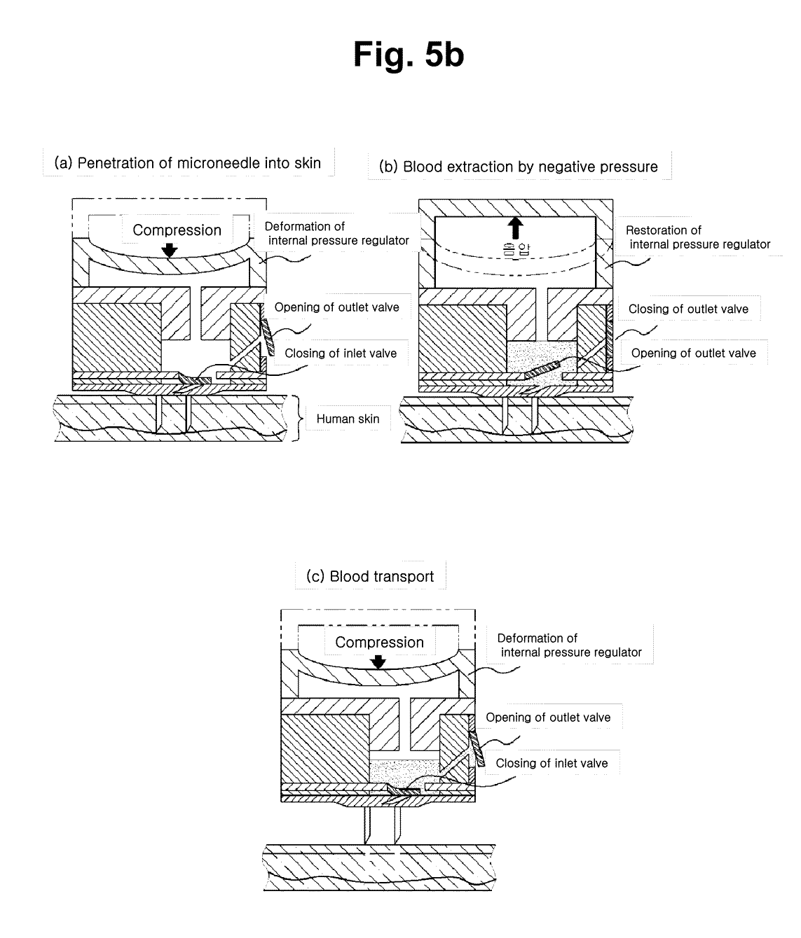

Then, the extracting performances of several fluids were evaluated using the devices for blood extraction of the present invention, which have internal pressure regulators with different volumes. As fluids, distilled water (DW), blood-mimicking fluid (BMF, blood-mimicking fluid in consideration of only the blood fluid without hemocytes (containing a ratio of 44:56 of glycerol: water, 15.68% sodium iodine salt; A Blood-mimicking fluid for particle image velocimetry with silicone vascular models, Experiments in Fluids, 50(3):1-6 (2010)), and human blood were used. As can be seen from FIG. 7, the larger the volume of the internal pressure regulator, the larger the extraction volume of fluids, that is, distilled water, BMF, and human blood, and thus, it can be seen that the device for blood extraction of the present invention was well operated.

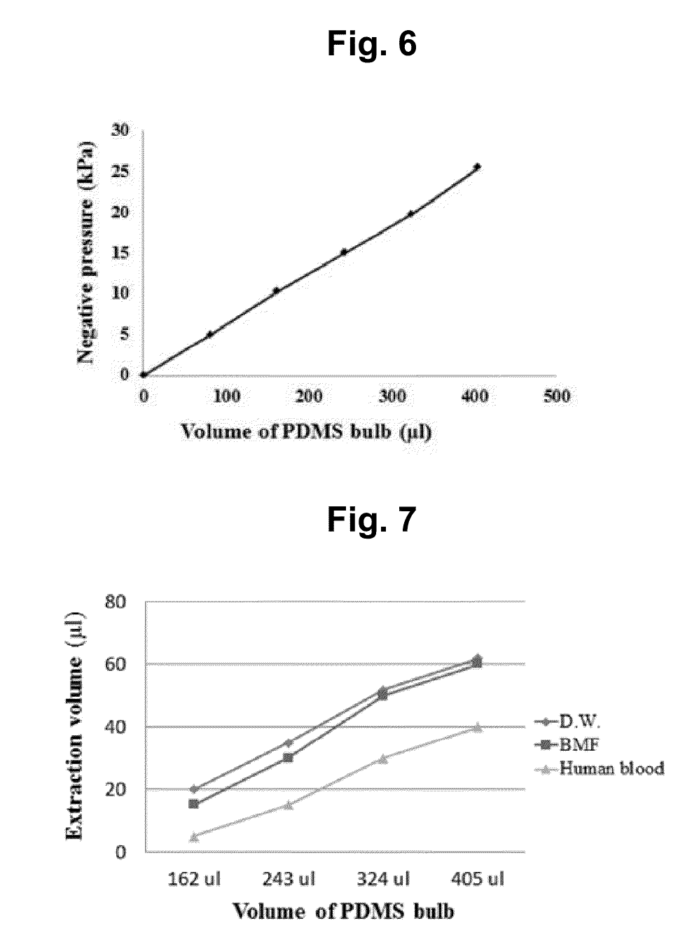

The mouse blood was extracted using the device for blood extraction of the present invention. The experiment was conducted using a hollow microneedle having a bevel angle of 15° and an inner diameter of 60 μm or 80 μm, and a 243-μl PDMS internal pressure regulator capable of producing an internal pressure of 14.95 kPa (FIG. 8). The device for blood extraction of the present invention was applied to the tail vein of an ICR mouse to extract blood twice therefrom, and the extraction volumes of blood were summarized in Table 2 below.

TABLE 2

| | Volume of | | |
|---|---|---|---|
| | internal | Extraction | |
| Dimension of | pressure | volume of | Extraction |
| microneedle | regulator | blood | time |
| 60-μm diameter and | 243 μl | 10 μl | 20 s |
| 15° bevel angle | | | |
| 80-μm internal | 243 μl | 20 μl | 25 s |
| diameter and 15° | | | |
| bevel angle | | | |

The blood from a rabbit (4 kg, New Zealand White) was extracted using the device for blood extraction of the present invention. The experiment was conducted using a hollow microneedle having a bevel angle of 15° and an inner diameter of 60 μm or 100 μm, and a 405-μl PDMS internal pressure regulator. The blood was extracted from upper, middle, and lower regions of the ear vein of the rabbit using the device for blood extraction of the present invention. The experiment was repeated three times. All experimental procedures were approved by the Department of Laboratory Animal Medicine, Yonsei University College of Medicine, and were performed in accordance with Animal Research Committee Guidelines at Yonsei University College of Medicine, approved by the AAALAC.

The device equipped with a 60-μm microneedle was used at an extraction rate of 3.1±0.2 μl s⁻¹ to extract 37.7±3.4 μl of blood, and the device equipped with a 100-μm microneedle was used at an extraction rate of 8.3±0.6 μl s⁻¹ to extract 124.5±5.1 μl of blood. The device equipped with a 60-μm microneedle extracted 37.7±3.4 μl of blood and transported 31.3±3.3 μl of blood. That is, the extracted blood was re-pressed by the PDMS internal pressure regulator 1 to obtain 31.3±3.3 μl of blood through the outlet valve 21, which corresponds to a sufficient volume for further microsystem analysis.

Therefore, it can be seen that the device for blood extraction of the present invention successfully performed in vivo blood extraction and transport.

### Example 6

### Incorporation of Device for Fluid Extraction (Blood Extraction) and Diagnostic Kit

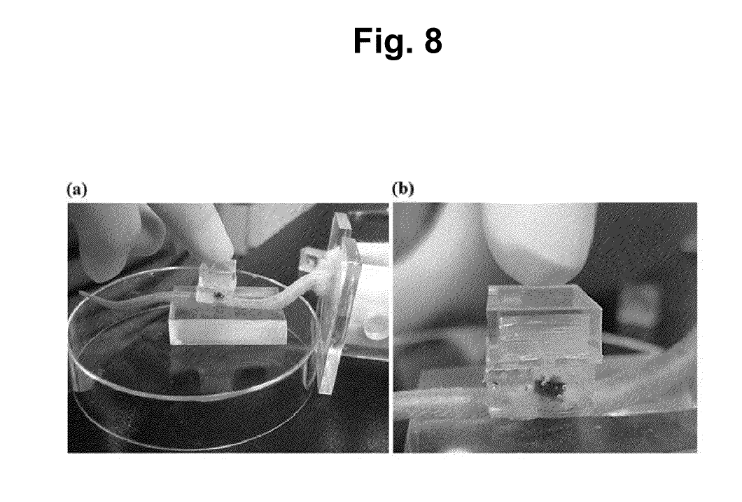

When the blood sample extracted using the device for blood extraction of the present invention is transported and loaded on a sample pad of a diagnostic kit through the outlet valve, a biosensor (e.g., an immunoassay kit combined with antibody) fixed to the diagnostic kit generates a signal, resulting in qualitative analysis or quantitative analysis of particular materials in the blood sample (FIG. 9). An integrated analysis system can be organized in such a manner.

### Example 7

### Integration of Device for Fluid Extraction (Blood Extraction) and Microchip

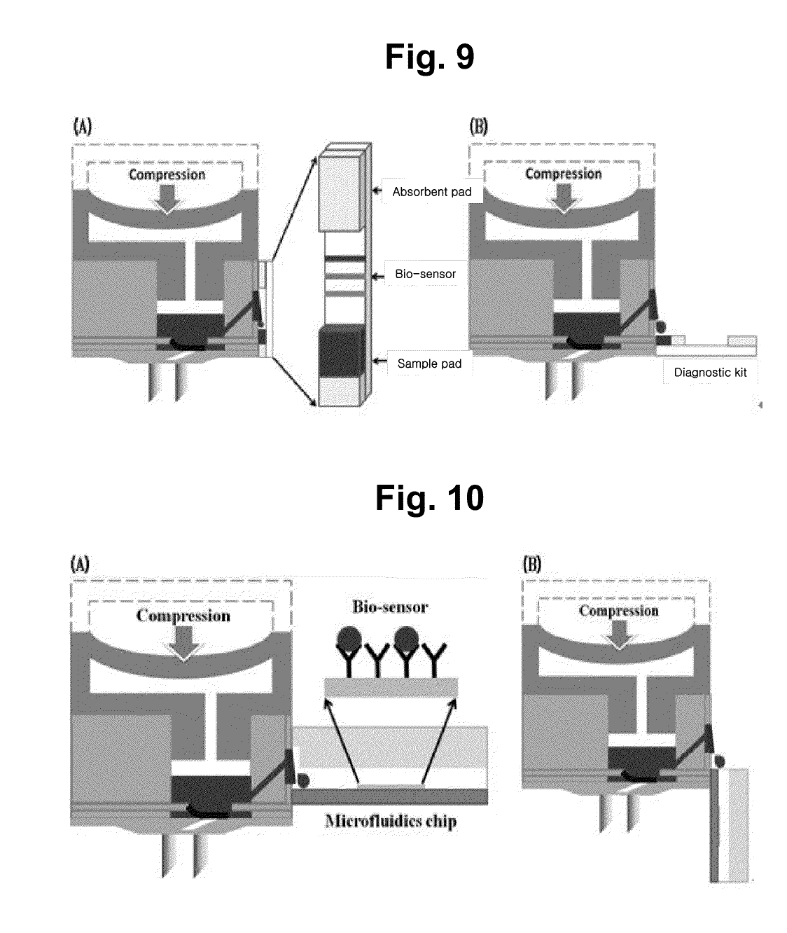

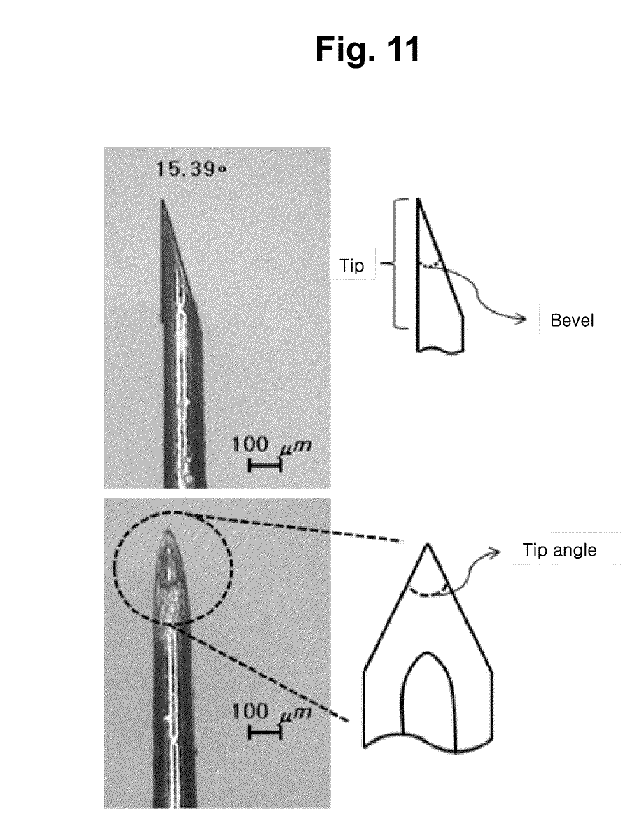

When the blood extracted by the device for blood extraction of the present invention is transported and loaded in a microchannel of a microchip through the outlet valve, a biosensor fixed to the microchannel generates a signal, resulting in qualitative analysis or quantitative analysis of particular materials in the blood sample (FIG. 10). An integrated analysis system can be organized in such a manner.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### REFERENCES

[1] Griss P, Stemme G. “Side-Opened Out-of-Plane Microneedles for Microfluidic Transdermal Liquid Transfer” (2003), J Microelectromech Syst 12, 296-301.
[2] Kazuyoshi Tsuchiya, Satoshi Jinnin, Hidetake Yamamoto, Yasutomo Uetsuji and Eiji Nakamachi. “Design and development of a biocompatible painless microneedle by the ion sputtering deposition method” (2010), Precision Engineering 34, 461-466.
[3] Sang Jun Moon, Seung S. Lee, H. S. Lee, T. H. Kwon. “Fabrication of microneedle array using LIGA and hot embossing process” (2005), Microsystem Technologies 11, 311-318.
[4] Giorgio E. Gattiker, Karan V. I. S. Kaler and Martin P. Mintchev. “Electronic Mosquito: designing a semi-invasive Microsystem for blood sampling, analysis and drug delivery applications” (2005), Microsystem Technologies 12, 44-51
[5] Kazuyoshi Tsuchiya, Naoyuki Nakanishi, Yasutomo Uetsuji, and Eiji Nakamachi. “Development of blood extraction system for health monitoring system” (2005), Biomedical Microdevices 7:4, 347-353.
[6] Nakanishi, N. Yamamoto, H. Tsuchiya, K. Uetsuji, Y. Nakamachi, E. “Development of Wearable medical device for bio-MEMS” (2006), BioMEMS and Nanotechnology II 6036, 168-176.
[7] Tsuchiya, K. Shimazu, Y. Uetsuji, Y. Nakamachi, E. “Development of blood extraction pump by shape memory alloy actuator for bio-MEMS” (2006), Biomedical Applications of Micro- and Nanoengineering II 6416, 64160A.
[8] K. Fujioka, S. Khumpuang, M. Horade and S. Sugiyama, Novel pressure-gradient driven component for blood extraction, to be published in Proc. of SPIE Microelectronics, MEMS, Nanotech., Brisbane, Australia, Dec. 11-15, 2005
[9] Design and fabrication of integrated passive valves and pumps for flexible polymer 3-Dimensional microfluidic systems, Noo Li Jeon, Daneil T. Chiu, Christopher J. Wargo, Hongkai Wu, Insung S. Choi, Janelle R. Anderson, and George M. Whitesides, 2002, Biomedical Microdevices 4:2, 117-121.
[10] B. H. Jo, L. M. V. Lerberghe, J. N. Motsegood, D. J. Beebe, “Three-dimensionalmicro-channel fabrication in poly-dimethylsiloxane (PDMS) elastomer” (2000), J. Microelectromech. Syst. 9, 76-81.
[11] Yang B and Lin Q, “Planar micro-check valves exploiting large polymer compliance” (2007), Sensors Actuators A 134, 186-93.
[12] Junhui Ni et al. “A planar PDMS micropump using in-contact minimized-leakage check valves” (2010), J. Micromech. Microeng. 20, 095033.
[13] Majid Y. Yousif•David W. Holdsworth•Tamie L. Poepping. “A blood-mimicking fluid for particle image velocimetry with silicone vascular models” (2011), Exp Fluids 50:3, 769-774.

1. A device for extraction of body fluids, the device comprising:
(a) an internal pressure regulator made of an elastically deformable material and having an internal space therein, the internal pressure regulator regulating the internal pressure of the device;
(b) a fluid reservoir openly connected to the internal pressure regulator and receiving body fluids extracted from the body; and
(c) a perforator connected to the fluid reservoir and disposed at a lower portion of the device, the perforator including a hollow microstructure that forms a hole in the body.

2. The device of claim 1, further comprising an outlet valve connected to the fluid reservoir and an inlet valve provided between the fluid reservoir and the hollow microstructure.

3. The device of claim 1, wherein the elastically deformable material is an epoxy polymer, a silicon polymer, or an acrylic polymer.

4. The device of claim 1, wherein the internal pressure regulator is deformed from its shape by a downward external pressure, resulting in reducing the volume of the internal space, thereby applying a perforation force to allow the hollow microstructure in contact with a body surface barrier to perforate the body surface barrier.

5. The device of claim 2, wherein the internal pressure regulator is deformed from its shape by a downward external pressure, resulting in reducing the volume of the internal space and thus increasing in the internal pressure of the device, thereby inducing the opening of the outlet value and the closing of the inlet valve and applying a perforation force to allow the hollow microstructure in contact with a body surface barrier to perforate the body surface barrier.

6. The device of claim 4, wherein the internal pressure regulator is restored to its original shape by the relaxing of the external pressure, resulting in producing a negative pressure inside the device, thereby allowing a fluid to flow into the fluid reservoir from the hollow microstructure in contact with the body surface barrier.

7. The device of claim 5, wherein the internal pressure regulator is restored to its original shape by the relaxing of the external pressure, resulting in producing a negative pressure inside the device and thus closing the outlet valve and opening the inlet valve, thereby allowing a fluid to flow into the fluid reservoir from the hollow microstructure in contact with the body surface barrier through the opened inlet valve.

8. The device of claim 6, wherein the internal pressure regulator after the relaxing of the external pressure is deformed from its shape by an application of a downward external pressure, resulting in reducing the volume of the internal pressure and thus increasing the internal pressure of the internal space, thereby allowing the fluid reserved in the fluid reservoir to flow out through the hollow microstructure.

9. The device of claim 7, wherein the internal pressure regulator after the relaxing of the external pressure is deformed from its shape by an application of a downward external pressure, resulting in reducing the volume of the internal pressure and thus increasing the internal pressure of the internal space, thereby inducing the opening of the outlet valve and the closing of the inlet valve and allowing the fluid reserved in the fluid reservoir to flow out through the outlet valve.

10. The device of claim 2, wherein the outlet valve, the inlet valve, or both of the outlet valve and the inlet valve have a pneumatic flap valve type.

11. The device of claim 2, wherein the outlet valve has an in-contact flap-stopper structure.

12. The device of claim 11, wherein the in-contact flap-stopper structure includes (i) an outlet valve flap plate having a flap that is openable and closable; and (ii) a stopper plate closely adhering to the outlet valve flap plate, a pore of the stopper plate communicating with the flap of the outlet valve flap plate when the flap is opened and the fluid reservoir.

13. The device of claim 2, wherein the inlet valve has a not-contact flap-stopper structure.

14. The device of claim 13, wherein the not-contact flap-stopper structure includes (i) an inlet valve flap plate having a flap that is openable and closable; (ii) a stopper plate having a pore communicating with the hollow microstructure; and (iii) an intermediate plate disposed between the inlet valve flap plate and the stopper plate, a pore of the intermediate plate communicating with the flap of the inlet valve flap plate when the flap is opened and the pore of the stopper plate.

15. The device of claim 1, wherein the hollow microstructure is a hollow microstructure for minimally invasive blood extraction that has a length of 200-5000 μm, an inner diameter of 10-100 μm, a bevel angle of 5-60°, a tip angle of 1-45°, and a tip transverse length of 2-30 μm.

16. An integrated analysis system of body fluids, the system comprising the device for extraction of body fluids of claim 1; and an analysis device of the body fluids.
